(19) United States
(12) Patent Application Publication
(10) Pub. No.: US 2024/0398481 A1
(43) Pub. Date: Dec. 5, 2024

(54) BONE REAMER VIDEO BASED NAVIGATION

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Brian William QUIST, Salem, NH (US); Paul Alexander TORRIE, Marblehead, MA (US); Nathan Anil NETRAVALI, Littleton, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 18/697,037

(22) PCT Filed: Nov. 30, 2022
(86) PCT No.: PCT/US2022/080660

§ 371 Date: Mar. 29, 2024

Related U.S. Application Data
(60) Provisional application No. 63/284,821, filed on Dec. 1, 2021.

(51) Int. Cl. A61B 34/10; A61B 34/20; A61B 90/00; A61B 90/96

(52) U.S. Cl. CPC A61B 34/10; A61B 34/20; A61B 90/06; A61B 90/37; A61B 90/96; A61B 2034/102; A61B 2034/105; A61B 2034/2055; A61B 2034/2065; A61B 2090/062

(57) ABSTRACT

In one embodiment of the present disclosure a method may include reading, by a surgical controller through an arthroscope, a portion of a machine-readable pattern on a reamer visible within a surgical site. The method may include determining, by the surgical controller, an actual depth of the reamer within a bone based on the portion of the machine-readable pattern. The method may include displaying, by the surgical controller on a display device, a value indicative of the actual depth of the reamer within the bone. The method may include controlling, based on the value of the actual depth, operation of a drill associated with the reamer.

### CROSS-REFERENCE TO RELATED APPLICATIONS

This application claim priority to and the benefit of U.S. Provisional Patent Application Ser. No. 63/284,821, filed Dec. 1, 2021, titled “Bone Reamer Video Based Navigation”, the entire disclosure of which is hereby incorporated by reference for all purposes.

### BACKGROUND

The anterior cruciate ligament (ACL) serves as the primary mechanical restraint in the knee to resist anterior translation of the tibia relative to the femur. Similarly the posterior cruciate ligament (PCL) serves as a mechanical restraint to resist posterior translation of the tibia relative to the femur. These cruciate ligaments contribute significantly to knee stability, and ACL injury is quite common. Most ACL injuries are complete tears of the ligament.

As ACL injuries occur often in patients that are young and active, reconstruction of the ACL is performed to enable return to activity. The goal is to restore stability of the knee and reduce the chances of further damage to the meniscus and articular cartilage that may lead to degenerative osteoarthritis. Reconstruction may consist of placement of a substitute graft (e.g., autograft from either the central third of the patellar tendon or the hamstring tendons). The ends of the graft are placed into respective tunnels prepared through the femur and the tibia. The ends of the graft may be attached using interference screws or a suspensory fixation device like the ENDOBUTTON™ brand fixation devices manufactured by Smith & Nephew of Andover, Massachusetts, USA.

One step in performing reconstruction surgery of the ACL includes drilling a femoral socket with a reamer. For certain types of fixation devices (e.g., adjustable loop or continuous loop), it may be desirable to achieve a specific target socket depth. Drilling a femoral socket that is too deep may create a thin bone wall too close to a cortex. As a result, in some instances, the thin bone wall may cause a failure of the ACL reconstruction.

### SUMMARY

In some examples, a method includes reading, by a surgical controller through an arthroscope, a portion of a machine-readable pattern on a reamer visible within a surgical site; determining, by the surgical controller, an actual depth of the reamer within a bone based on the portion of the machine-readable pattern; and displaying, by the surgical controller on a display device, a value indicative of the actual depth of the reamer within the bone.

In some examples, a surgical controller includes a processor configured to couple to a display device; a memory coupled to the processor, the memory storing instructions that, when executed by the processor, cause the processor to: receive one or more images of a visible portion of a reamer used at a surgical site for a bone, wherein the one or more images comprise a portion of a machine-readable pattern included on the reamer; determine, based on the portion of the machine-readable pattern included on the reamer, a value indicative of a parameter associated with the reamer; display, on a display device, the value indicative of the parameter.

In some examples, an intraoperative method includes displaying, by a surgical controller on a display device, a value indicative of a parameter associated with a reamer visible within a surgical site; determining whether the value indicative of the parameter satisfies a threshold value for the parameter; and responsive to determining the value indicative of the parameter satisfies the threshold value for the parameter, controlling operation of a drill associated with the reamer at the surgical site by modifying an operating parameter of the drill.

In some examples, a surgical controller includes a processor configured to couple to a display device; a memory coupled to the processor, the memory storing instructions that, when executed by the processor, cause the processor to: obtain, via an arthroscope, one or more images of a portion of a machine-readable pattern on a reamer at a surgical site; determine, based on the images, coordinates of a location of the machine-readable pattern within a three-dimensional coordinate space; determine, based on the coordinates of the location and bone geometry information mapped to the three-dimensional coordinate space, an actual depth of the reamer within a bone at the surgical site; and display, on the display device, a value indicative of the actual depth of the reamer.

### BRIEF DESCRIPTION OF THE DRAWINGS

For a detailed description of example embodiments, reference will now be made to the accompanying drawings in which:

FIG. 1 shows an anterior or front elevation view of right knee, with the patella removed;

FIG. 2 shows a posterior or back elevation view of the right knee;

FIG. 3 shows a view of the femur from below and looking into the intercondylar notch;

FIG. 4 shows a surgical system in accordance with at least some embodiments;

FIG. 5 shows a conceptual drawing of a surgical site with various objects within the surgical site tracked, in accordance with at least some embodiments;

FIG. 6 is an example video display showing portions of a femur and having visible therein a bone fiducial, in accordance with at least some embodiments;

FIG. 7 is an example video display showing intraoperative changes to a tunnel path, in accordance with at least some embodiments;

FIG. 8 is an example reamer including a machine-readable pattern, in accordance with at least some embodiments;

FIG. 9 is an example video display showing a portion of a machine-readable pattern of a reamer inserted in a bone, in accordance with at least some embodiments;

FIG. 10 is an example video display for configuring a target depth of a tunnel path, in accordance with at least some embodiments;

FIG. 11 is an example method for intraoperative determination of a depth of a reamer within a bone, in accordance with at least some embodiments;

FIG. 12 is an example method for controlling a drill based on a determined depth of a reamer within a bone, in accordance with at least some embodiments;

FIG. 13A is an example method for determining a depth of a reamer based on a coordinate space, in accordance with at least some embodiments; and

FIG. 13B is an example method for reducing a field of view to determine a depth of a reamer based on a coordinate space, in accordance with at least some embodiments; and

FIG. 14 shows a computer system in accordance with at least some embodiments.

### DEFINITIONS

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct connection or through an indirect connection via other devices and connections.

An endoscope having “a single optical path” through an endoscope shall mean that the endoscope is not a stereoscopic endoscope having two distinct optical paths separated by an interocular distance at the light collecting end of the endoscope. The fact that an endoscope has two or more optical members (e.g., glass rods, optical fibers) forming a single optical path shall not obviate the status as a single optical path.

“Throughbore” shall mean an aperture or passageway through an underlying device. However, the term “throughbore” shall not be read to imply any method of creation. Thus, a throughbore may be created in any suitable way, such as drilling, boring, laser drilling, or casting.

“Counterbore” shall mean an aperture or passageway into an underlying device. In cases in which the counterbore intersects another aperture (e.g., a throughbore), the counter bore may thus define an internal shoulder. However, the term “counterbore” shall not be read to imply any method of creation. A counterbore may be created in any suitable way, such as drilling, boring, laser drilling, or casting.

The terms “input” and “output” when used as nouns refer to connections (e.g., electrical, software), and shall not be read as verbs requiring action. For example, a timer circuit may define a clock output. The example timer circuit may create or drive a clock signal on the clock output. In systems implemented directly in hardware (e.g., on a semiconductor substrate), these “inputs” and “outputs” define electrical connections. In systems implemented in software, these “inputs” and “outputs” define parameters read by or written by, respectively, the instructions implementing the function.

“Controller” shall mean, alone or in combination, individual circuit components, an application specific integrated circuit (ASIC), a microcontroller with controlling software, a reduced-instruction-set computing (RISC) with controlling software, a digital signal processor (DSP), a processor with controlling software, a programmable logic device (PLD), or a field programmable gate array (FPGA), configured to read inputs and drive outputs responsive to the inputs.

“Real-time” may refer to an action, operation, method, function, instruction, etc. being performed within 2 seconds. “Near real-time” may refer to an action, operation, method, function, instruction, etc. being performed between 2 seconds and 1 minute.

### DETAILED DESCRIPTION

The following discussion is directed to various embodiments of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Various examples are directed to methods and systems of ligament reconstruction. The ligament repair (e.g., anterior cruciate ligament (ACL) repair) may be performed arthroscopically and computer assisted. Some examples include methods and systems of using an endoscopic optical system comprising an arthroscope and attached camera head. The endoscopic optical system may be used to read at least a portion of a machine-readable pattern on a reamer attached to a drill as the reamer drills a hole in a bone. A surgical controller may receive the read portion of the machine-readable pattern (e.g., one or more images) and may determine one or more parameters associated with the reamer, the bone, the drill, or some combination thereof. For example, the surgical controller may determine one or more parameters including an actual depth of the reamer within a bone at a surgical site, a diameter of the reamer, an identifier of the reamer, or some combination thereof. The surgical controller may control operation of a drill associated with the reamer based on the one or more parameters. In some embodiments, a notification may be depicted on a display device when a threshold depth value is about to be reached or has been exceeded.

The various examples were developed in the context of ACL repair, and thus the discussion below is based on the developmental context. However, the techniques are applicable to many types of ligament repair, such as medial collateral ligament repair, lateral collateral ligament repair, and posterior cruciate ligament repair. Moreover, the various example methods and systems can also be used for planning and placing anchors to reattach soft tissue, such as reattaching the labrum of the hip, the shoulder, or the meniscal root. Thus, the description and developmental context shall not be read as a limitation of the applicability of the teachings. In order to orient the reader, the specification first turns a description of the knee.

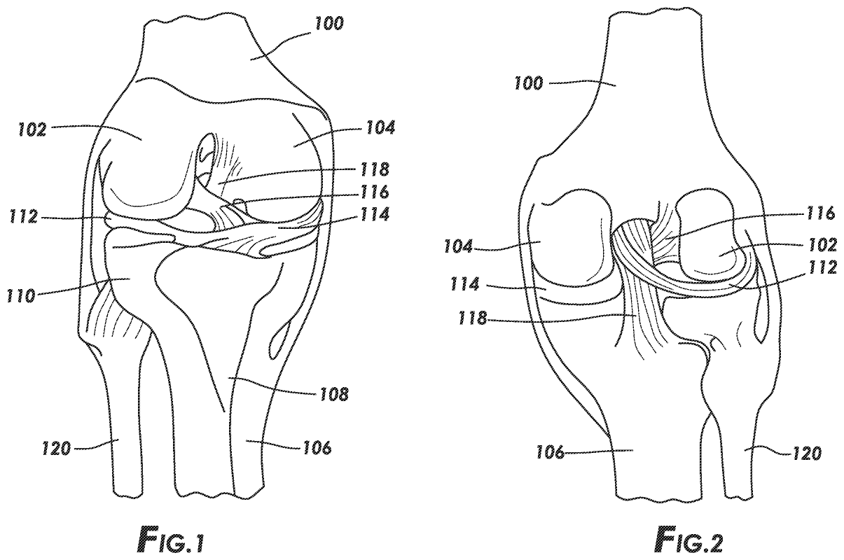

FIG. 1 shows an anterior or front elevation view of a right knee, with the patella removed. In particular, visible in FIG. 1 is lower portion of the femur 100 including the outer or lateral condyle 102 and the inner or medial condyle 104. The femur 100 and condyles 102 and 104 are in operational relationship to a tibia 106 including the tibial tuberosity 108 and Gerdy's tubercle 110. Disposed between the femoral condyles 102 and 104 and the tibia 106 are the lateral meniscus 112 and the medial meniscus 114. Several ligaments are also visible in the view of FIG. 1, such as the ACL 116 extending from the lateral side of femoral notch to the medial side of the tibia 106. Oppositely, the posterior cruciate ligament 118 extends from medial side of the femoral notch to the tibia 106. Also visible is the fibula 120, and several additional ligaments that are not specifically numbered.

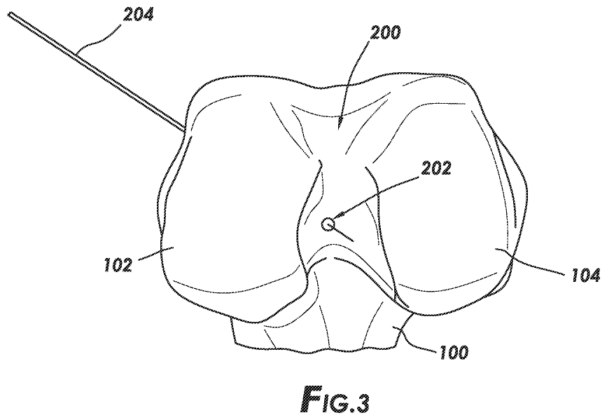

FIG. 2 shows a posterior or back elevation view of the right knee. In particular, visible in FIG. 2 is lower portion of the femur 100 including the lateral condyle 102 and the medial condyle 104. The femur 100 and femoral condyles 102 and 104 again are in operational relationship to the tibia 106, and disposed between the femoral condyles 102 and 104 and the tibia 106 are the lateral meniscus 112 and the medial meniscus 114. FIG. 2 further shows the ACL 116 extending from the lateral side of femoral notch to the medial side of the tibia 106, though the attachment point to the tibia 106 is not visible. The posterior cruciate ligament 118 extends from medial side of the femoral notch to the tibia 106, though the attachment point to the femur 100 not visible. Again several additional ligaments are shown that are not specifically numbered.

The most frequent ACL injury is a complete tear of the ligament. Treatment involves reconstruction of the ACL by placement of a substitute graft (e.g., autograft from either the patellar tendon, quad tendon, or the hamstring tendons). The graft is placed into tunnels prepared within the femur 100 and the tibia 106. The current standard of care for ACL repair is to locate the tunnels such that the tunnel entry point for the graft is at the anatomical attachment location of the native ACL. Such tunnel placement at the attachment location of the native ACL attempts to recreate original knee kinematics. In arthroscopic surgery, the location of the tunnel through the tibia 106 is relatively easy to reach, particularly when the knee is bent or in flexion. However, the tunnel through the femur 100 resides within the intercondylar notch. Depending upon the physical size of the patient and the surgeon's selection for location of the port through the skin, and through which the various instruments are inserted into the knee, it may be difficult to reach the attachment location of the native ACL to the femur 100.

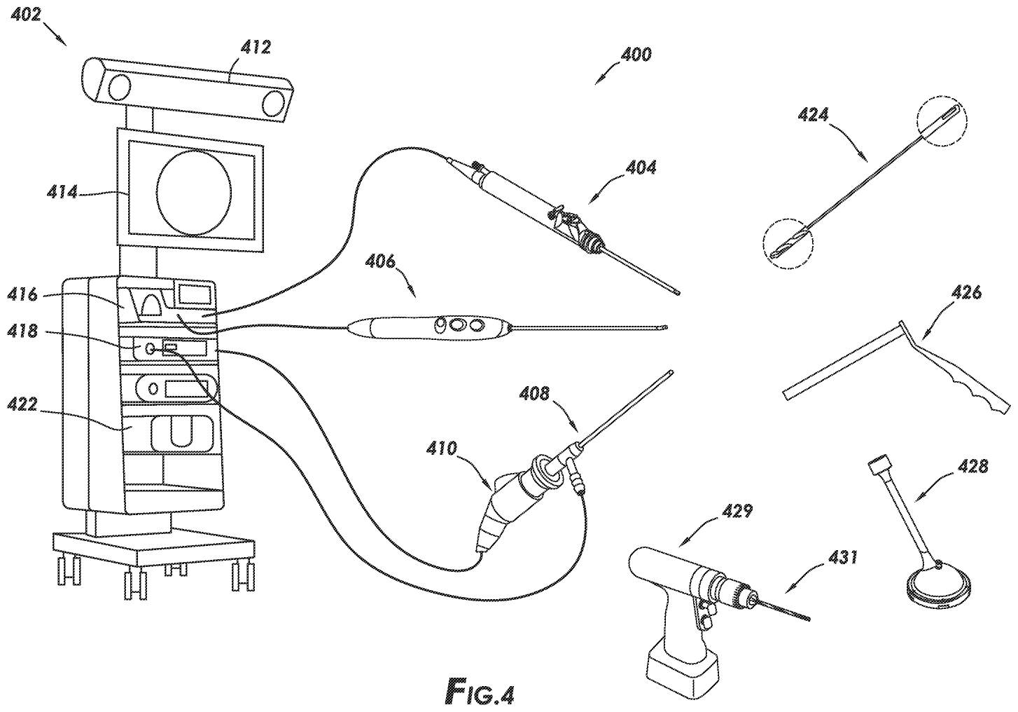

FIG. 3 shows a view of the femur from below and looking into the intercondylar notch. In particular, visible in FIG. 3 are the lateral condyle 102 and the medial condyle 104. Defined between the femoral condyles 102 and 104 is the femoral notch 200. The femoral tunnel may define inside aperture 202 within the femoral notch 200, the inside aperture 202 closer to the lateral condyle 102 and displaced into the posterior portion of the femoral notch 200. The femoral tunnel extends through the femur 100 and forms an outside aperture on the outside or lateral surface of the femur 100 (the outside aperture not visible in FIG. 3). FIG. 3 shows an example drill wire 204 that may be used to create an initial tunnel or pilot hole. Once the surgeon verifies that the drill wire 204 is closely aligned with a planned-tunnel path, the femoral tunnel is created by boring or reaming with another instrument (e.g., a reamer) that may use the drill wire 204 as a guide. In some cases, a socket or counterbore is created on the intercondylar notch side to accommodate the width of the graft that extends into the bone, and that counterbore may also be created using another instrument (e.g., reamer) that may use the drill wire 204 as a guide.

Drilling of a tunnel may take place from either direction. Considering the femoral tunnel again as an example, the tunnel may be drilled from the outside or lateral portion of the femur 100 toward and into the femoral notch 200, which is referred to as an “outside-in” procedure. Oppositely, the example femoral tunnel may be drilled from the inside of the femoral notch 200 toward and to the lateral portion of the femur 100, which is referred as an “inside-out” procedure. The various examples discussed below are equally applicable to outside-in or inside-out procedures. Outside-in procedures may additionally use a device which holds the drill wire on the outside portion, and physically shows the expected tunnel location of the inside aperture within the knee. However, the device for the outside-in procedure is difficult to use in arthroscopic procedures, and thus many arthroscopic repairs use the inside-out procedure. The further examples discussed below are thus based on an inside-out procedure, but such should not be read as a limitation. The specification now turns to an example surgical system.

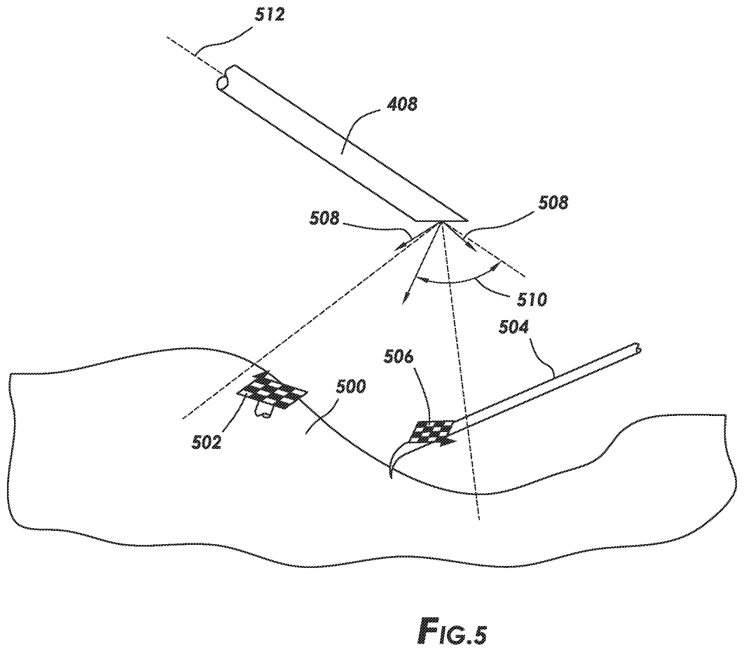

FIG. 4 shows a surgical system (not to scale) in accordance with at least some embodiments. In particular, the example surgical system 400 comprises a tower or device cart 402, an example mechanical resection instrument 404, an example plasma-based ablation instrument (hereafter just ablation instrument 406), and an endoscope in the example form of an arthroscope 408 and attached camera head 410. The device cart 402 may comprise a camera 412 (illustratively shown as a stereoscopic camera), a display device 414, a resection controller 416, and a camera control unit (CCU) together with an endoscopic light source and video controller 418. In example cases the CCU and video controller 418 not only provides light to the arthroscope 408 and displays images received from the camera head 410, but also implements various additional aspects, such as determining values of one or more parameters associated with a reamer (e.g., actual depth of reamer within a bone, diameter of reamer, identifier of reamer, etc.), displaying values of the one or more parameters on the display device 414, receiving input to modify an operating parameter (e.g., revolutions per minute, power on/off, drilling direction, etc.) of a drill associated with the reamer, controlling operation of a drill based on the one or more parameters, and so forth. Thus, the CCU and video controller is hereafter referred to as surgical controller 418. In other cases, however, the CCU and video controller may be a separate and distinct system from the controller that handles aspects of intraoperative changes, yet the separate devices would nevertheless be operationally coupled.

The example device cart 402 further includes a pump controller 422 (e.g., single or dual peristaltic pump). Fluidic connections of the mechanical resection instrument 404 and ablation instrument 406 are not shown so as not to unduly complicate the figure. Similarly, fluidic connections between the pump controller 422 and the patient are not shown so as not to unduly complicate the figure. In the example system, both the mechanical resection instrument 404 and the ablation instrument 406 are coupled to the resection controller 416 being a dual-function controller. In other cases, however, there may be a mechanical resection controller separate and distinct from an ablation controller. The example devices and controllers associated with the device cart 402 are merely examples, and other examples include vacuum pumps, patient-positioning systems, robotic arms holding various instruments, ultrasonic cutting devices and related controllers, patient-positioning controllers, and robotic surgical systems.

FIG. 4 further shows additional instruments that may be present during an example ACL repair. In particular, FIG. 4 shows an example guide wire or drill wire 424 and an aimer 426. The drill wire 424 may be used to create an initial or pilot tunnel through the bone. In some cases, the diameter of the drill wire may be about 2.4 millimeters (mm), but larger and smaller diameters for the drill wire 424 may be used. The example drill wire 424 is shown with magnified portions on each end, one to show the cutting elements on the distal end of the drill wire 424, and another magnified portion to show a connector for coupling to chuck of a drill. Once the surgeon drills the pilot tunnel, the surgeon and/or the surgical controller 418 may then assess whether the pilot tunnel matches or closes matches the planned-tunnel path. If the pilot tunnel is deemed sufficient, then the drill wire 424 may be used as a guide for creating the full-diameter throughbore for the tunnel, or alternatively, for creating a counterbore stemming from the intercondylar notch to accommodate the graft. While in some cases the drill wire alone may be used when creating the pilot tunnel, in yet still other cases the surgeon may use the aimer 426 to help guide and place the drill wire 424 at the designed tunnel-entry location.

FIG. 4 also shows that the example system may comprise a calibration assembly 428. As will be discussed in greater detail below, the calibration assembly 428 may be used to detect optical distortion in images received by the surgical controller 418 through the arthroscope 408 and attached camera head 410.

Additional tools and instruments will be present, such as a drill 429 for drilling with the drill wire 424, various reamers 431 for creating the throughbore and counterbore aspects of the tunnel, and various tools for suturing and anchoring the graft in place. Some of these additional tools and instruments are not shown so as not to further complicate the figure.

The specification now turns to a workflow for an example ACL repair. The workflow may be conceptually divided into a preoperative planning and intraoperative repair. The intraoperative repair workflow may be further conceptually divided into optical system calibration, model registration, intraoperative tunnel-path planning, intraoperative and tunnel creation. Each will be addressed in turn.

### Preoperative Planning

In accordance with various examples, an ACL repair starts with preoperative imaging (e.g., X-ray imaging, computed tomography (CT), magnetic resonance imaging (MRI)) of the knee of the patient, including the relevant anatomy like the lower portion of the femur, the upper portion of the tibia, and the articular cartilage. The discussion that follows assumes MRI imaging, but again many different types of preoperative imaging may be used. The MRI imaging can be segmented from the image slices such that a volumetric model or three-dimensional model of the anatomy is created. Any suitable currently available, or after developed, segmentation technology may be used to create the three-dimensional model. More specifically to the example of ACL repair and specifically selecting a tunnel path through the femur, a three-dimensional bone model of the lower portion of the femur, including the femoral condyles, is created.

Using the three-dimensional bone model, an operative plan is created that comprises choosing a planned-tunnel path through the femur, including locations of the apertures of the bone that define the ends of the tunnel. For an example inside-out repair, the aperture within the femoral notch is the entry location for the drilling, and the aperture on the lateral surface of the femur is the exit location. For an outside-in repair, the entry and exit locations for drilling are swapped. Still assuming an inside-out repair, the entry location may be selected to be the same as, or close to, the attachment location of the native ACL to the femur within the femoral notch. In some cases, selecting the entry location within the femoral notch may involve use of a Bernard & Hertel Quadrant or grid placed on a fluoroscopic image, or placing the Bernard & Hertel Quadrant on a simulated fluoroscopic image created from the three-dimensional bone model. Based on use of the Bernard & Hertel Quadrant, an entry location for the tunnel is selected. For an inside-out repair, selection of the exit location is less restrictive, not only because the portion of the tunnel proximate to the exit location is used for placement of the anchor for the graft, but also because the exit location is in approximately centered in the femur (considered anteriorly to posteriorly), and thus issues of bone wall thickness at the exit location are of less concern. In some cases, a three-dimensional bone model of the proximal end of the tibia is also created, and the surgeon may likewise choose planned-tunnel path(s) through the tibia.

The results of the preoperative planning may comprise: a three-dimensional bone model of the distal end of the femur; a three-dimensional bone model for a proximal end of the tibia; an entry location and exit location through the femur and thus a planned-tunnel path for the femur; and an entry location and exit location through the tibia and thus a planned-tunnel path through the tibia.

In some embodiments, using the three-dimensional bone model, the surgeon may select a planned value for a socket or counterbore depth of a reamer into a bone. The surgeon may select the socket or counterbore depth such that a socket is not created that is too long, thereby resulting in a thin bone wall that may be too close to the cortex. A bone wall that is thin and too close to the cortex may result in a failure of the ACL reconstruction. In addition, using the three-dimensional bone model, the surgeon may select a tunnel throughbore diameter, a counterbore diameter, and the like. One or more reamers may be selected based on the diameters of the tunnel throughbore diameter and/or the counterbore diameter.

In some embodiments, the three-dimensional bone model may include a virtual representation of the imaged bones of the patient mapped into a three-dimensional coordinate space (e.g., virtual space). The dimensions, size, density, length, width, depth, height, shape, position, configuration, orientation, and the like of the bones may be accurately represented in the three-dimensional coordinate space.

Other surgical parameters may also be selected during the preoperative planning, such as desired post-repair flexion, and the like, but those additional surgical parameters are omitted so as not to unduly complicate the specification.

### Intraoperative Repair

The specification now turns to intraoperative aspects. The intraoperative aspects include steps and procedures for setting up the surgical system to perform the various repairs. It is noted, however, that some of the intraoperative aspects (e.g., optical system calibration), may take place before any ports or incisions are made through the patient's skin, and in fact before the patient is wheeled into the surgical room. Nevertheless, such steps and procedures are considered intraoperative as they take place in the surgical setting and with the surgical equipment and instruments used to perform the actual repair.

The example ACL repair is conducted arthroscopically and is computer-assisted in the sense the surgical controller 418 is used for arthroscopic navigation within the surgical site. More particularly, in example systems the surgical controller 418 provides computer-assistance during the ligament repair by tracking location of various objects within the surgical site, such as the location of the bone within the three-dimensional coordinate space of the view of the arthroscope, the location of the reamer within the three-dimensional coordinate space of the view of the arthroscope, and location of the various instruments (e.g., the drill wire 424, the aimer 426) within the three-dimensional coordinate space of the view of the arthroscope. The specification turns to brief description of such tracking techniques.

FIG. 5 shows a conceptual drawing of a surgical site with various objects within the surgical site. In particular, visible in FIG. 5 is a distal end of the arthroscope 408, a portion of a bone 500 (e.g., femur), a bone fiducial 502 within the surgical site, a touch probe 504, and a probe fiducial 506. Each is addressed in turn.

The distal end of the arthroscope 408 is designed and constructed to illuminate the surgical site with visible light. In the example of FIG. 5, the illumination is illustrated by arrows 508. The illumination provided to the surgical site is reflected by various objects and tissues within the surgical site, and the reflected light that returns to the distal end enters the arthroscope 408, propagates along an optical channel within the arthroscope 408, and is eventually incident upon a capture array within the camera head 410 (FIG. 4). The images detected by the capture array within the camera head 410 are sent electronically to the surgical controller 418 (FIG. 4) and displayed on the display device 414 (FIG. 4). In accordance with example systems, the arthroscope 408 has a single optical path through the arthroscope for capturing images of the surgical site, notwithstanding that the single optical path may be constructed of two or more optical members (e.g., glass rods, optical fibers). That is to say, in example systems and methods the computer-assisted navigation provided by the arthroscope 408, camera head 410, and surgical controller 418 is provided with the arthroscope 408 that is not a stereoscopic endoscope having two distinct optical paths separated by an interocular distance at the distal end endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as “zero degree” arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30° arthroscope the viewing direction forms a 30° angle to the longitudinal central axis of the arthroscope, the angle measured as an obtuse angle beyond the distal end of the arthroscope. In many cases for ACL repair, the surgeon selects a 30° arthroscope or a 45° arthroscope based on location the port created through the skin of the patient. In the example of FIG. 5, the view angle 510 of the arthroscope 408 forms a non-zero angle to the longitudinal central axis 512 of the arthroscope 408.

Still referring to FIG. 5, within the view of the arthroscope 408 is a portion of the bone 500, along with the bone fiducial 502, the touch probe 504, and the probe fiducial 506. The bone fiducial 502 is shown as a planar element having a pattern disposed thereon, though other shapes for the bone fiducial 502 may be used (e.g., a square block with a pattern on each face of the block). The bone fiducial 502 may be attached to the bone 500 in any suitable form (e.g., a fastener, such as a screw). The pattern of the bone fiducial is designed to provide information regarding the orientation of the bone fiducial 502 in the three-dimensional coordinate space of the view of the arthroscope 408. More particularly, the pattern is selected such that the orientation of the bone fiducial 502, and thus the orientation of the underlying bone 500, may be determined from images captured by the arthroscope 408 and attached camera head 410 (FIG. 4).

The probe fiducial 506 is shown as a planar element attached to the touch probe 504. The touch probe 504 may be used, as discussed more below, to “paint” the surface of the bone 500 as part of the registration of the bone 500 to the three-dimensional bone model, and the touch probe 504 may also be used to indicate revised-tunnel entry locations in the case of intraoperative changes to the tunnel paths. The probe fiducial 506 is shown as a planar element having a pattern disposed thereon, though other shapes for the probe fiducial 506 may be used (e.g., a square block surrounding the touch probe 504 with a pattern on each face of the block). The pattern of the probe fiducial 506 is designed to provide information regarding the orientation of the probe fiducial 506 in the three-dimensional coordinate space of the view of the arthroscope 408. More particularly, the pattern is selected such that the orientation of the probe fiducial 506, and thus the location of the point of the touch probe 504, may be determined from images captured by the arthroscope 408 and attached camera head 410 (FIG. 4).

Other instruments within the view of the arthroscope 408 may also have fiducials, such as the drill wire 424 (FIG. 4) and aimer 426 (FIG. 4), but the additional instruments are not shown so as not unduly complicate the figure. Moreover, in addition to or in place of tracking location based on the view through the arthroscope 408, the location of the distal end of one or more of the instruments may be tracked by other methods and systems. For example, for devices that rigidly extend out of the surgical site (e.g., the aimer 426 (FIG. 4)), the location may be tracked by an optical array coupled to the aimer and viewed through the camera 412 (FIG. 4), such as a stereoscopic camera. The location within the three-dimensional coordinate space of the camera 412 is then transformed into the three-dimensional coordinate space of the view of the example arthroscope to determine location of the distal end within the surgical site.

The images captures by the arthroscope 408 and attached camera head 410 are subject to optical distortion in many forms. For example, the visual field between distal end of the arthroscope 408 and the bone 500 within the surgical site is filled with fluid, such as bodily fluids and saline used to distend the joint. Many arthroscopes have one or more lenses at the distal end that widen the field of view, and creating wider field of view causes a “fish eye” effect in the captured images. Further, the optical elements within the arthroscope (e.g., rod lenses) may have optical aberrations inherent to the manufacturing and/or assembly process. Further still, the camera head 410 may have various optical elements for focusing the images receives onto the capture array, and the various optical elements may have aberrations inherent to the manufacturing and/or assembly process.

### Optical System Calibration

In example systems, prior to use within each surgical procedure, the endoscopic optical system is calibrated to account for the various optical distortions. In particular, various embodiments comprise a system for calibrating the endoscopic optical system. Referring again to FIG. 4, the example system comprises the surgical controller 418, the arthroscope 408, the camera head 410, and the calibration assembly 428. In particular, the calibration may comprise placing the arthroscope 408 into the calibration assembly 428. The calibration assembly 428 holds the arthroscope 408 in a fixed relationship to a calibration target on an inside surface of the calibration assembly 428. Once the distal end of the arthroscope 408 is within the calibration assembly 428, the example method comprises capturing a plurality of images of the calibration target, with each image captured at a unique rotational relationship between the camera head 410 and the arthroscope 408, with the unique rotational relationships relative to a longitudinal central axis of the arthroscope 408. Using the plurality of images, the example surgical controller 418 creates a characterization function that characterizes optical distortion between the calibration target and the capture array within the camera head 410. The characterization function may include a calibration for determining orientation of fiducial markers visible within the surgical site (e.g., bone fiducial 502, probe fiducial 506) by way of the arthroscope 408 and attached camera head 410.

### Model Registration and Human-In-the-Loop Registration Verification

The next example step in the intraoperative procedure is the registration of the bone model(s) created during the preoperative planning. That is, during the preoperative planning stage, preoperative imaging (e.g., MRI) of the knee takes place, including the relevant anatomy like the lower portion of the femur, the upper portion of the tibia, and the articular cartilage. The preoperative imaging can be segmented such that a volumetric model or three-dimensional model of the anatomy is created. More specifically to the example of ACL repair, and specifically selecting a tunnel path through the femur, a three-dimensional bone model of the lower portion of the femur is created during the preoperative planning. Further, a depth of the tunnel path may be selected and a diameter of the tunnel may be selected. The depth and diameter may be represented in the volumetric or three-dimensional model (e.g., a virtual space including axes and coordinates (e.g., X, Y, Z)).

During the intraoperative repair, the three-dimensional bone models are provided to the surgical controller 418. Again using the example of ACL repair, and specifically computer-assisted navigation for tunnel paths through the femur, the three-dimensional bone model of the lower portion of the femur is provided to the surgical controller 418. Thus, the surgical controller 418 receives the three-dimensional bone model, and assuming the arthroscope 408 is inserted into the knee by way of a port through the patient's skin, the surgical controller 418 also receives video images of the femur. In order to relate the three-dimensional bone model to the images received by way of the arthroscope 408 and camera head 410, the surgical controller 418 registers the three-dimensional bone model to the images of the femur received by way of the arthroscope 408 and camera head 410.

In accordance with example methods, a fiducial marker or bone fiducial (e.g., bone fiducial 502 of FIG. 5) is attached to the femur. The bone fiducial placement is such that the bone fiducial is within the field of view of the arthroscope 408, but in a location spaced apart from the expected tunnel entry/exit point through the lateral condyle. More particularly, in example cases the bone fiducial is placed within the intercondylar notch superior to or above the expected location of the tunnel through lateral condyle.

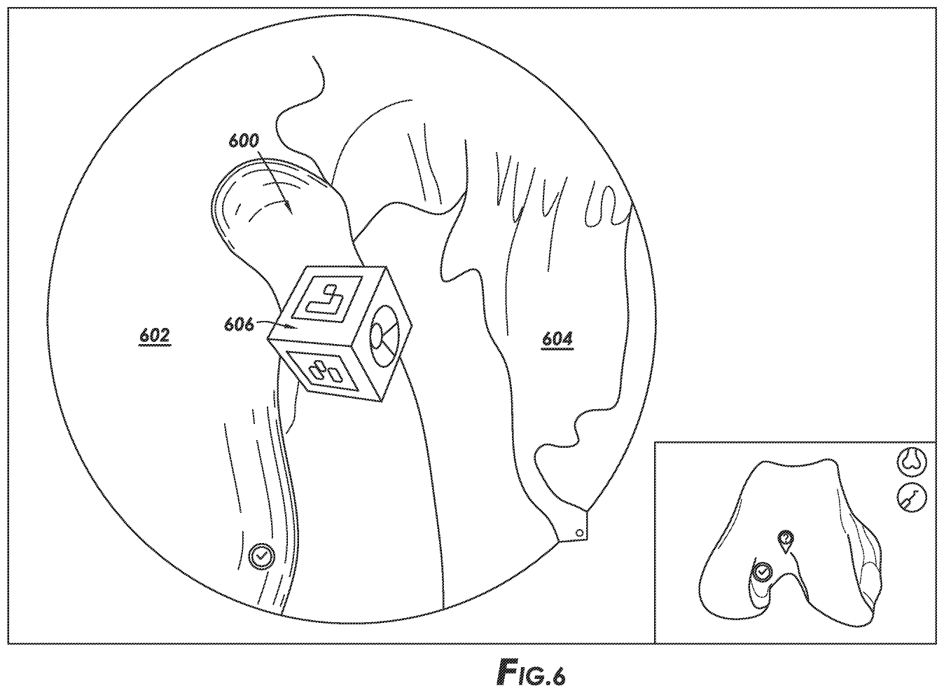

FIG. 6 is an example video display showing portions of a femur and a bone fiducial. The display may be shown, for example, on the display device 414 (FIG. 4) associated with the device cart 402 (FIG. 4), or any other suitable location. In particular, visible in FIG. 6 is a femoral notch or intercondylar notch 600, a portion of the lateral condyle 602, a portion the medial condyle 604, and an example bone fiducial 606. The bone fiducial 606 is a fiducial comprising a cube member. Of the six outer faces of the cube member, the bottom face is associated with an attachment feature (e.g., a screw). The bottom face will be close to or abut the bone when the bone fiducial 606 is secured in place, and thus will not be visible in the view of the arthroscope 408 (FIG. 4). The outer face opposite the bottom face includes a placement feature used to hold the bone fiducial 606 prior to placement, and to attach the bone fiducial 606 to the underlying bone. Of the remaining four outer faces of the cube member (only two of the remaining faces are visible), each of the four outer faces has a machine-readable pattern thereon, and in some cases each machine-readable pattern is unique. Once placed, the bone fiducial 606 represents a fixed location on the outer surface of the bone in the view of the arthroscope 408, even as the position of the arthroscope 408 is moved and changed relative to the bone fiducial 606. Initially, the location of the bone fiducial 606 with respect to the three-dimensional bone model is not known to the surgical controller 418, hence the need for the registration of the three-dimensional bone model.

In or order to relate or register the bone visible in the video images to the three-dimensional bone model, the surgical controller 418 (FIG. 4) is provided and thus receives a plurality of locations of an outer surface of the bone. For example, the surgeon may touch a plurality of locations using the touch probe 504 (FIG. 5). As previously discussed, the touch probe 504 comprises a probe fiducial 506 (FIG. 5) visible in the video images captured by the arthroscope 408 (FIG. 4) and camera head 410 (FIG. 4). The physical relationship between the distal end of the touch probe 504 and the probe fiducial 506 is known by the surgical controller 418, and thus as the surgeon touches each of the plurality of locations on the outer surface of the bone, the surgical controller 418 gains an additional “known” locations of the outer surface of the bone relative to the bone fiducial 606. Given that the touch probe 504 is a relatively inflexible instrument, in other examples the tracking of the touch probe 504 may be by optical tracking of an optically-reflective array outside the surgical site (e.g., tracking by the camera 412 (FIG. 4)) yet attached to the portion of the touch probe 504 inside the surgical site.

In some cases, particularly when portions of the outer surface of the bone are exposed to view, receiving the plurality of locations of the outer surface of the bone may involve the surgeon “painting” the outer surface of the bone. “Painting” is a term of art that does not involve application of color or pigment, but instead implies motion of the touch probe 504 when the distal end of the touch probe 504 is touching bone. In some embodiments, other modes of capturing the shape of the bone relative to the bone fiducial 606 may include using computed tomography (CT) and/or fluoroscopy with or without contrast (e.g., a substance that is injected into an intravenous line or taken by mouth to enable the body part (e.g., bone tissue and structure) being examined to be seen more clearly). CT scans may use an X-ray beam that traverses a body part externally in a circular motion to obtain many different views of the same body part and those images may be used to generate a two-dimensional form of the body part on a video display. Fluoroscopy may refer to imaging technique that uses a fluoroscope to obtain real-time moving images of a body part of the patient. CT and/or fluoroscopy may enable obtaining updated images of the outer surface of the bone relative to the bone fiducial 606. In some embodiments, another mode of capturing the shape of the bone relative to the bone fiducial 606 may include using an externally tracked ultrasound probe. An external tracker may be used to identify a position and orientation of images obtained by an ultrasound probe that may be merged to identify the outer surface of the bone relative to the bone fiducial 606.

A video display may be displayed during a registration procedure showing portions of a femur, a bone fiducial, and an overlaid representation of the three-dimensional bone model. The display may be shown, for example, on the display device 414 (FIG. 4) associated with the device cart 402 (FIG. 4), or any other suitable location. In order to address potential invalid registrations between the three-dimensional bone model and the bone visible in the images, in example methods and systems an initial registration between the bone visible in the video images and the three-dimensional bone model is verified using a human-in-the-loop process. More particularly, in example cases, after receiving the plurality of locations of an outer surface of the bone visible in the images and performing an initial registration to the three-dimensional bone model, the surgical controller 418 displays a representation of the three-dimensional bone model overlaid on portions of the bone visible in the video images, the displaying and overlaying on the display device 414, or other suitable location. That is, the example surgical controller 418 overlays a representation of the three-dimensional bone model (e.g., by way of a polygon mesh, or mesh model) on the display of the images captured by the arthroscope 408 and camera head 410, wherein the rotational and translational alignment of the three-dimensional bone model is correlated to the bone visible in the video images as anchored by the bone fiducial 606.

The surgeon, in turn, visually studies the overlaid representation of the three-dimensional bone model relative to the underling bone visible in the video images to determine whether the registration process was correct. More particularly, the surgeon visually compares the overlaid representation of the three-dimensional bone model to the portion of the bone visible in the video images to determine whether the three-dimensional bone model sufficiently matches the bone visible in the video images. Much like the registration process itself, the human-in-the-loop verification of registration is a non-deterministic exercise. Slight variances between the three-dimensional bone model and the bone visible in the video images may be tolerated, yet nevertheless the registration process may be considered correct in the sense that the three-dimensional bone model may be reliably used to help guide placement of the tunnel path (e.g., here the femoral tunnel path), or assist the surgeon in intraoperative changes to the planned-tunnel path. In such cases, the surgeon may provide the surgical controller 418, and the surgical controller 418 may thus receive, an indication that the three-dimensional bone model is correctly registered to the bone visible in the video images.

On the other hand, if the overlay of the three-dimensional bone model shows misalignment with the bone visible in the video images, the surgeon may elect to restart the registration process, such as by providing to the surgical controller 418, and the surgical controller 418 receiving again, a plurality of locations of the outer surface of the bone. The surgical controller 418 may then perform anew the registration procedure. In other cases, the surgeon may elect to provide additional locations on the outer surface of the bone, and the surgical controller 418 may then perform the registration procedure with the both original locations received and the additional locations received after the overlay process. The process repeats until the surgeon approves the registration. The specification now turns to intraoperative tunnel path planning.

### Tunnel Path Planning

Using the three-dimensional bone model an operative plan is preoperatively created that comprises a planned-tunnel path through the bone, including locations of the apertures into the bone that define the ends of the tunnel. Further, a depth of the planned-tunnel path is selected and a diameter of the tunnel path is selected.

In some cases, however, the surgeon may elect not to use planned-tunnel path, and thus elect not use the preoperatively planned entry location, exit location, or both. Such an election can be based any of a number of reasons. For example, intraoperatively the surgeon may not be able to access the entry location for the planned-tunnel path, and thus may need to move the entry location to ensure sufficient access. As another example, during the intraoperative procedure the surgeon may determine that the planned tunnel entry location is misaligned with the attachment location of the native ACL to the femur. Further still, during the intraoperative procedure the surgeon may determine the tunnel entry location is too close to the posterior wall of the femur, increasing the likelihood of a bone chip sometimes referred to as a “back wall blowout.” Regardless of the reason for the election to change the tunnel path, in example systems the surgical controller 418 enables the surgeon to intraoperatively select a revised-tunnel entry, a revised-tunnel exit (if needed), and thus a revised-tunnel path through the bone.

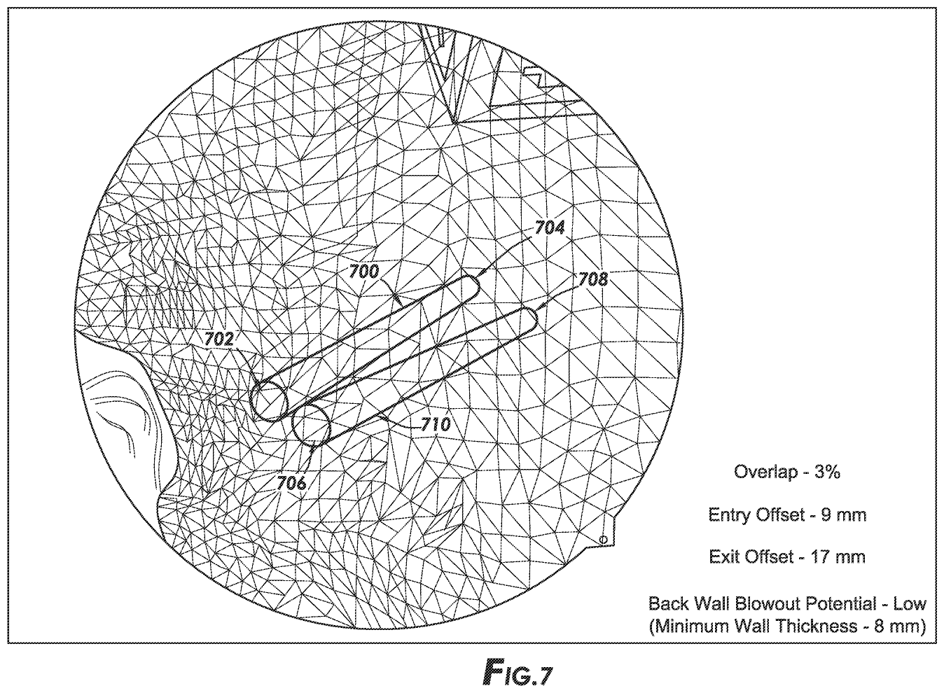

FIG. 7 is an example video display showing intraoperative changes to the tunnel path, in accordance with at least some embodiments. The display may be shown, for example, on the display device 414 (FIG. 4) associated with the device cart 402 (FIG. 4), or any other suitable location. In particular, FIG. 7 shows a portion of the bone in the video images as captured by the arthroscope 408 (FIG. 4) overlaid with the mesh model of the three-dimensional bone model and a preoperatively selected planned-tunnel path 700 comprising a planned-tunnel entry 702 and a planned-tunnel exit 704. However, for any number of reasons, the surgeon may elect to modify the tunnel entry location and/or the tunnel exit location, and thus modify the planned-tunnel path. Thus, the surgeon may provide to the surgical controller 418 (FIG. 4), and thus the surgical controller 418 may receive, a revised-tunnel entry location or just revised-tunnel entry 706. Providing the revised-tunnel entry 706 may comprise the surgeon touching the proposed location on the bone shown in the video images with a tracked tool, such as the touch probe 504 (FIG. 5) or the aimer 426 (FIG. 4). In some cases, the surgeon may select the revised-tunnel entry 706 based solely on what the surgeon sees of the bone shown in the video images. As a more specific example, the surgeon may select and provide the revised-tunnel entry 706 based on a location that can be reached by the aimer 426. In yet still other cases, the surgical controller 418 may generate a simulated fluoroscopic images from the three-dimensional bone model, and project thereon a Bernard & Hertel Quadrant or grid. The surgeon may then select the revised-tunnel entry 706 with the additional guidance provided by the Bernard & Hertel Quadrant.

### Tunnel Creation

With the revised-tunnel path 710 selected, the next step in the example method is creation of the actual tunnel. In most cases, creating the tunnel is a multistep process involving drilling an initial or pilot tunnel using a drill wire (e.g., drill wire 424 (FIG. 4)), and then using the drill wire as guide wire for one or more reamers to increase the diameter of the pilot tunnel to form the full-diameter actual tunnel though the bone. In some cases the actual tunnel has a counterbore associated with the intercondylar notch to accommodate the width of the autograft, and in such cases an additional reamer may be used to create the counterbore.

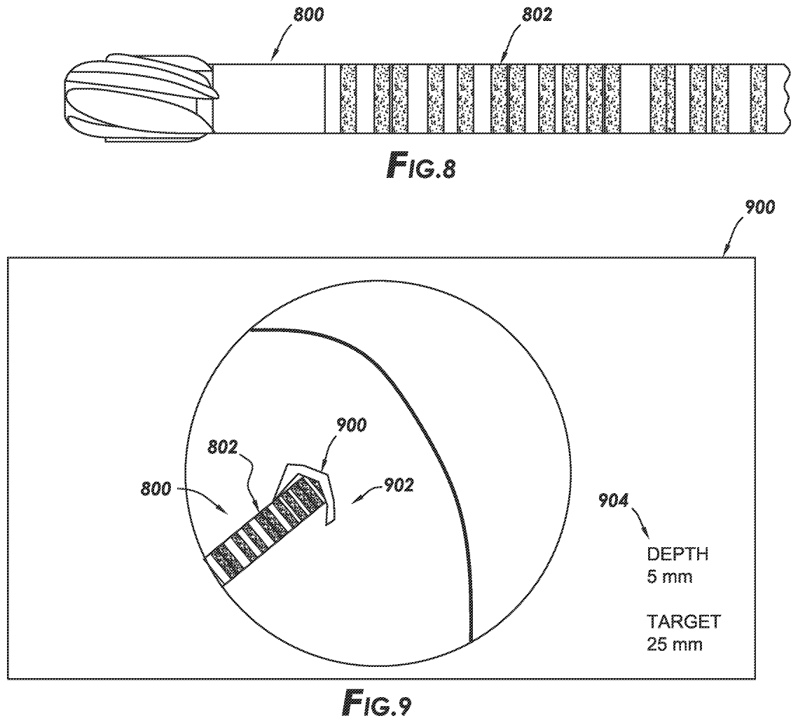

FIG. 8 is an example reamer 800 including a machine-readable pattern 802, in accordance with at least some embodiments. The depicted machine-readable pattern 802 includes a set of markings that are radially continuous around the reamer 800. The markings have variable thicknesses. The spacing between the markings and the widths of the markings may be correlated with a value at each portion of the reamer. The value may be interpreted by a processor of the surgical controller 418 as the reamer 800 spins during drilling to determine a depth of the reamer 800 within the bone. Although the markings are depicted as being one or more annular stipes that circumscribe the reamer 800, in some examples, the markings may be one or more annular stripes that partially circumscribes the reamer 800.

In some embodiments, the markings may be engraved or notched on the reamer 800 at a smaller diameter than the diameter of the portion of a shaft of the reamer 800 that includes cutting flutes. Such a technique may enable minimizing or reducing the degradation of the markings when the reamer 800 is being drilled into a bone.

Further, the arthroscope and camera may view the reamer 800 while it's spinning or rotating during drilling and may transmit one or more images to the surgical controller 418. The surgical controller 418 may receive the images in real-time or near real-time and determine a value indicative of a parameter encoded by the markings of the machine-readable pattern 802. In some embodiments, just a side of the reamer 800 may be visible by the arthroscope and camera during drilling and the machine-readable pattern 802 may enable determining a depth of the reamer in the bone at a continuous or continual pace while drilling. In some examples, the markings may be akin to a barcode that is interpreted by the surgical controller 418 during drilling to determine a depth of the reamer 800 within the bone in real-time or near real-time. In some examples, the reamer 800 may be spinning at a high revolutions per minute, which may cause motion blurring to occur in the images that are processed. The markings may be configured to generate a gradient as the reamer 800 is spinning and the gradient may be different at portions along a length of the reamer 800. Each gradient may be correlated with a certain depth value, and the surgical controller 418 may determine the depth of the reamer within the bone based on the gradients.

In some embodiments, the surgical controller 418 may restrict the field of view of the one or more images to a location known to include the reamer 800 inserted into the bone. For example, the surgical controller 418 may register the three-dimensional bone models with the one or more images of the surgical site as previously described herein. Thus, the surgical controller 418 may identify the longitudinal central axis of the arthroscope and the drill wire to focus the image processing on a smaller portion of the one or more images than captured by the camera head. Such a technique may reduce the file size of the one or more images that are processed by the surgical controller 418, thereby enhancing the use of processing and memory resources and providing a technical improvement. Based on the three-dimensional models, images, drill wire, and/or axis of the arthroscope, the surgical controller 418 may crop the one or more images to just include the insertion point of the reamer 800 into the bone. The other portions of the one or more images not in proximity to the insertion point of the reamer 800 into the bone may be discarded and/or deleted. The surgical controller 418 may determine a depth of the reamer 800 within the bone along an axis known (e.g., identified in a model such as the bone model and/or a drilling plan) to the surgical controller 418 and while the reamer 800 is rotating during drilling of the bone.

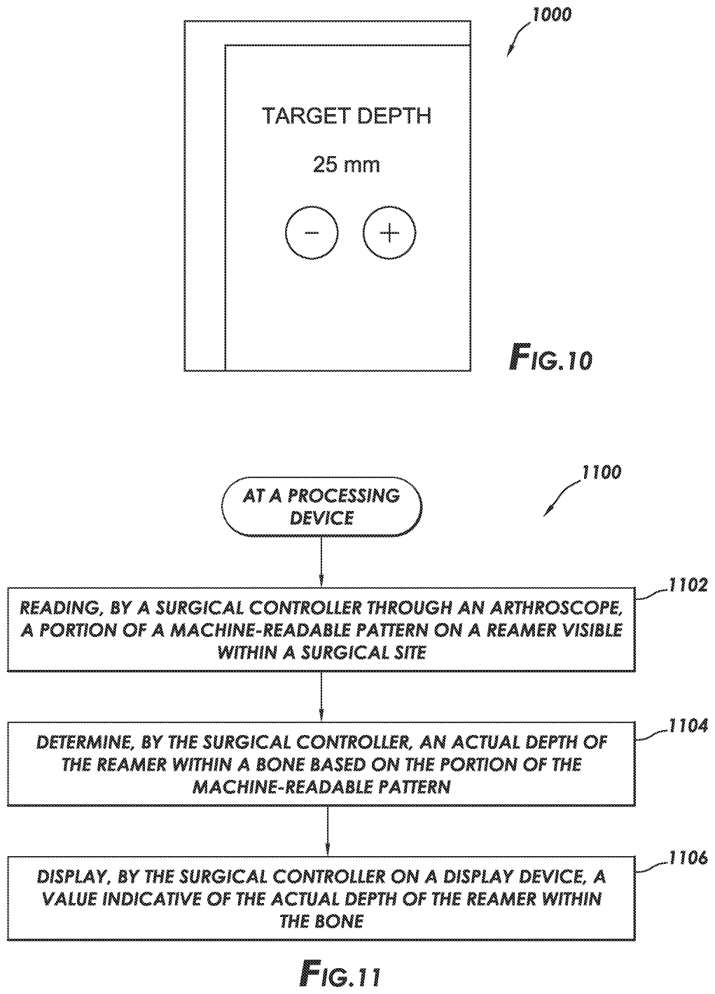

FIG. 9 is an example video display 900 showing a portion of a machine-readable pattern 802 of a reamer 800 inserted in a bone 902, in accordance with at least some embodiments. The video display 900 may be presented by display device 414 of the device cart and/or a computing device (e.g., smartphone, tablet, laptop, etc.) associated with the device cart. The video display 900 enables a surgeon to view the reamer 800 as it drills the tunnel path. Further, the video display 900 includes graphical elements 904 that depict the current depth (e.g., actual depth of 5 millimeters) of the reamer 800 within the bone 902 in real-time or near real-time. Further, the graphical elements 904 depict a target depth (e.g., 25 millimeters) of a socket or counterbore, and the target depth may be configured by the surgeon in a preoperative phase or may be set by default. In some embodiments, the graphical elements 904 may depict a diameter of the reamer 800 as determined by the surgical controller 418 reading a portion of the machine-readable pattern 802. In some examples, the graphical elements 904 may depict an identification of the reamer 800 as determined by the surgical controller 418 reading a portion of the machine-readable pattern 802.

FIG. 10 is an example video display 1000 for configuring a target depth of a tunnel path, in accordance with at least some embodiments. The example video display 1000 may be provided by a computing device (e.g., smartphone, tablet, laptop, etc.) associated with the device cart. For example, the video display 1000 may be provided by a tablet used by a user to control the target depth. The target depth may be configured during the preoperative phase of the medical procedure. As depicted, the surgeon may select various graphical elements to increase or decrease a target depth of a tunnel path. The target depth may be used as a threshold value by the surgical controller 418 to determine whether to slow down operation of the drill, stop operation of the drill, increase operation of the drill, or the like. That is, the actual depth of the reamer 800 within the bone 902, as determined by the surgical controller 418 reading a portion of the machine-readable pattern 802, may be compared to the target depth, and the comparison may result in the surgical controller 418 electronically controlling operation of the drill. The surgical controller 418 may enable an operator to continue drilling if desired.

### Software and Hardware

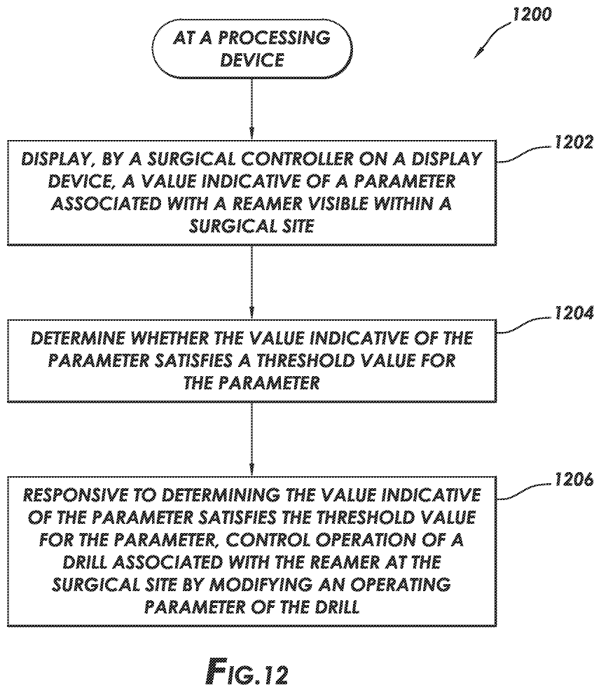

FIG. 11 shows a method of intraoperative determination of an actual depth of a reamer within a bone, in accordance with at least some embodiments. In particular, the methods starts at block 1100. The method may be performed by one or more processors, such as included in the surgical controller 418, for example. The method may be implemented in computer instructions stored in one or more memories that are executed by the one or more processors.

At block 1102, the processor of the surgical controller 418 may read, using an arthroscope and a camera, a portion of a machine-readable pattern 802 on a reamer 800 visible within a surgical site. The surgical controller 418 may receive one or more images of the visible portion of the reamer 800 used at the surgical site of the bone 902. In some embodiments, the portion of the machine-readable pattern 802 may include at least one annular stripe that circumscribes the reamer 800. In some embodiments, the portion of the machine-readable pattern 802 may include one marking that only partially circumscribes the reamer 800. In some embodiments, the machine-readable pattern 802 may include a set of radial markings along a central axis of the reamer 800. In some embodiments, the set of radial markings may be spaced apart by a distance representative of a value of the actual depth. The markings may be configured such that the spinning of the reamer 800 causes a visual pattern to be generated that is interpreted by the surgical controller 418 to determine a depth of the reamer 800 within the bone 902. In one example, the machine-readable pattern 802 may be human-readable (e.g., numbers) to interpret the location/depth of the reamer 800 within the bone in addition to being machine-readable to enable two options for determining the location/depth of the reamer 800.

In some embodiments, a first portion of a shaft of the reamer 800 including the machine-readable pattern 802 may have a smaller diameter than a second portion of the shaft of the reamer 800 including cutting flutes. That is, the portion of the shaft including the machine-readable pattern may be “necked down”. Such a technique may minimize or reduce pattern erosion due to bone friction when using the reamer 800 to drill a tunnel in a bone 902. In some embodiments, a robotic arm may support positioning of the reamer 800 as it drillings the tunnel in the bone 902.

In some embodiments, prior to reading the portion of the machine-readable pattern 802 on the reamer 800, the surgical controller 418 may reduce a revolutions per minute of the reamer from a first value to a second value. For example, during drilling operation, a drill may rotate the reamer 800 at a high number of revolutions per minute. The high speed rotations may cause motion blur in images captured by the camera. In some instances, the revolutions per minute of the reamer may be reduced to a threshold number such that the machine-readable pattern 802 may be clearly read.

In some embodiments, the portion of the machine-readable pattern 802 may include at least one marking including a barcode encoding the value of the actual depth of the reamer 800 within the bone 902. The barcode may be included along a length of the reamer 800. The barcode may include one or more complete or partial annular markings having width spacing and/or thicknesses that encode and indicate a value of an actual depth of the reamer 800 within the bone 902. For example, the surgical controller 418 may read the last visible portion of the barcode at an entry point to the bone and determine, based on the barcode, the value of the reamer 800 within the bone 902.

In some embodiments, the machine-readable pattern 802 may include a set of tick marks that are spaced apart along the length of the reamer 800. A tick mark may refer to a visual marking on the shaft of the reamer 800, where the visual marking fully or partially circumscribes the reamer 800. The visual marking may include a value, color, shape, letter, or some combination thereof. In some examples, the visual marking may include an etched mark on the shaft of the reamer 800. In some examples, each visual marking representing each tick mark may be different or the same. Each tick mark may be correlated with a particular depth of a distal end of the reamer 800 within a bone 902. In some embodiments, the tick marks may be numbered on the reamer 800. The smallest value for a tick mark that is visible near an entry point to the bone 902 may be selected as the depth of the reamer 800 within the bone 902. The value of the tick mark may be searched in a lookup table to determine an associated actual depth value for that tick mark. In some embodiments, the tick marks may be counted by the surgical controller 418 as the reamer 800 is inserted within the bone 902. The number of tick marks counted may indicate the depth of the reamer 800 within the bone 902.

In some embodiments, during drilling operations, the reamer 800 may be spinning at a high number of revolutions per minute (e.g., hundreds, thousands, ten-thousands, etc.) and the machine-readable pattern 802 may be affected by motion blurring. The surgical controller 418 may receive the images that are continuously or continually obtained by the camera through the arthroscope. When the reamer 800 is spinning at the high revolutions per minute, the machine-readable pattern 802 may be configured to produce a gradient having thicknesses, colors, tones, contrasts, sharpness, brightness, shades, or the like that indicate a value of a depth of a reamer 800 within a bone 902. A gradient may refer to a change in intensity or color of an image or a portion of an image (e.g., corresponding to a visual marking on the reamer 800) that is identified and processed by the surgical controller 418. The surgical controller 418 may identify a gradient produced by the machine-readable pattern 802 as the reamer 800 rotates. The surgical controller 418 may determine, based on the gradient and a data source, the actual depth of the reamer 800 within the bone 902. The data source may store information (e.g., thickness, color, tone, contrast, sharpness, brightness, shade, etc.) pertaining to various gradients and the information may be associated with a particular depth value of the reamer 800 when that particular gradient is detected.

In some embodiments, the processor of the surgical controller 418 may read the portion of the machine-readable pattern 802 at a first set of coordinates within a coordinate space. The processor may receive bone geometry information mapped within the coordinate space. The bone geometry information may be represented by the three-dimensional bone model generated and previously described herein. The bone geometry information may include information related to bone type, density, size, shape, length, location, orientation, configuration, state, and the like. The processor may determine, based on comparing the first set of coordinates and the bone geometry information, at least one of the actual depth of the reamer, an orientation of the reamer, and the trajectory of the reamer 800. For example, the length of a portion of the machine-readable pattern 802 and the distal end of the reamer 800 may be known by the surgical controller 418 and the coordinates of the portion of the machine-readable pattern 802 may enable determining the location of the reamer in the coordinate space. Further, the coordinates of the location of the desired tunnel and an entry point to the tunnel may be known by the surgical controller 418. Based on the coordinates of the portion of the machine-readable pattern 802 and the coordinates of the entry point to the tunnel, the surgical controller 418 may determine how far into the tunnel the reamer 800 is inserted based on its length and location relative to the entry point. Further, based on the coordinates of the portion of the machine-readable pattern 802 and the coordinates of the entry point to the tunnel, the surgical controller 418 may determine the orientation of the reamer 800 and/or the trajectory of the reamer 800 into the bone.

At block 1104, the processor of the surgical controller 418 may determine an actual depth of the reamer 800 within a bone 902 based on the portion of the machine-readable pattern 802.

In some embodiments, determining the actual depth of the reamer 800 may include extracting, by the surgical controller 418, a set of depth values from the portion of the machine-readable pattern 802. The surgical controller 418 may select only one of the set of depth values to be the actual depth.

In some embodiments, determining the actual depth of the reamer 800 within the bone may include using a temporal sequence guide associated with a set of markings comprising the machine-readable pattern 802. The temporal sequence guide may specify that readings of the machine-readable pattern 802 occur in a sequenced order from a distal end to a proximal end of the reamer 800, and that each portion of the machine-readable pattern 802 (e.g., markings) that is subsequently read in the sequenced order indicates the reamer 800 is further disposed within the bone 902 at a particular depth. The temporal sequence guide may be stored as a lookup table in a memory device and accessed by the surgical controller 418.

In some embodiments, one or more other parameters in addition to actual depth may be determined by the surgical controller 418 based on the portion of the machine-readable pattern 802. For example, a diameter of the reamer 800 may be encoded in the portion of the machine-readable pattern 802. Determining a diameter of the reamer 800 being used in real-time or near real-time may enable the surgeon to verify the right-sized reamer 800 is being using to drill a tunnel having a desired diameter.

In some embodiments, the one or more parameters may include an identifier of the reamer 800. For example, the identifier may be encoded in the portion of the machine-readable pattern 802. The identifier may be unique for each individual reamer.

In some examples, determining the value of a parameter (e.g., actual depth, diameter, identifier) may include using infrared navigation of a drill and an arthroscopic camera to triangulate a location of the reamer in three-dimensional coordinate space. Triangulating the location of the reamer 800 in three-dimensional coordinate space may include identifying positional coordinates of the drill and the arthroscopic camera and triangulating (e.g., using angles from the drill and the arthroscopic camera to the reamer and a distance between the camera and the arthroscopic camera) a point on the reamer 800 indicative of a depth of the reamer 800 within the bone 902.

Additionally, in another example, determining the value of the parameter may include using one or more three-dimensional positioning tools including a fiber optic shaped sensing (FOSS) system, an electromagnetic (EM) system, or some combination thereof. The three-dimensional positioning tools may be implemented at least partially as computer instructions executed by the surgical controller 418. For example, the FOSS system may include using a fiber optic cable and/or fiber optic sensor to continuously track the three-dimensional shape and position of the reamer 800. Light reflected off the reamer may be received in the arthroscope and transmitted to the surgical controller 418 to determine the shape and position of the reamer in the coordinate space. Based on the position of the reamer in the coordinate space, the surgical controller 418 may determine a depth of the reamer within a bone (e.g., using a bone model).

At block 1106, the processor of the surgical controller 418 may display on a display device 414 a value indicative of the actual depth of the reamer 800 within the bone 902. In some embodiments, the processor of the surgical controller 418 may display, in real-time or near real-time, on the display device 414 a value indicative of the diameter of the reamer 800, the identifier of the reamer 800, the actual depth of the reamer 800 within the bone, or some combination thereof. In some embodiments, the processor of the surgical controller 418 may control, based on the parameter (e.g., actual depth of the reamer within the bone, diameter of the reamer, identifier of the reamer) of the reamer 800, operation of the reamer 800 by modifying an operating parameter of a drill. For example, the operating parameter may pertain to a drilling direction, a rotational speed (e.g., revolutions per minute), an operating state (e.g., on or off), or some combination thereof.

In some embodiments, the surgical controller 418 may receive input (e.g., via an input peripheral such as a keyboard, mouse, microphone, touchscreen, etc.) pertaining to controlling the operation of the reamer 800. For example, a surgeon may view the actual depth of the reamer within the bone as compared to a target depth and may enter input that specifies slowing down drilling or stopping drilling such that the target depth is not exceeded. As a result, one or more command instructions may be transmitted from the surgical controller 418 to the drill to modify the operating parameter. The drill and the surgical controller 418 may be communicatively coupled to each other.

In some embodiments, the surgical controller 418 may determine, based on the value of the actual depth, whether a planned depth (e.g., target depth) of the reamer 800 within the bone has been satisfied. The surgical controller 418 may display a notification based on whether the value indicative of the actual depth of the reamer within the bone is within a threshold amount from the planned depth or has exceeded the planned depth. Responsive to determining the planned depth has been satisfied, the surgical controller 418 may electronically stop a drill operating the reamer 800.

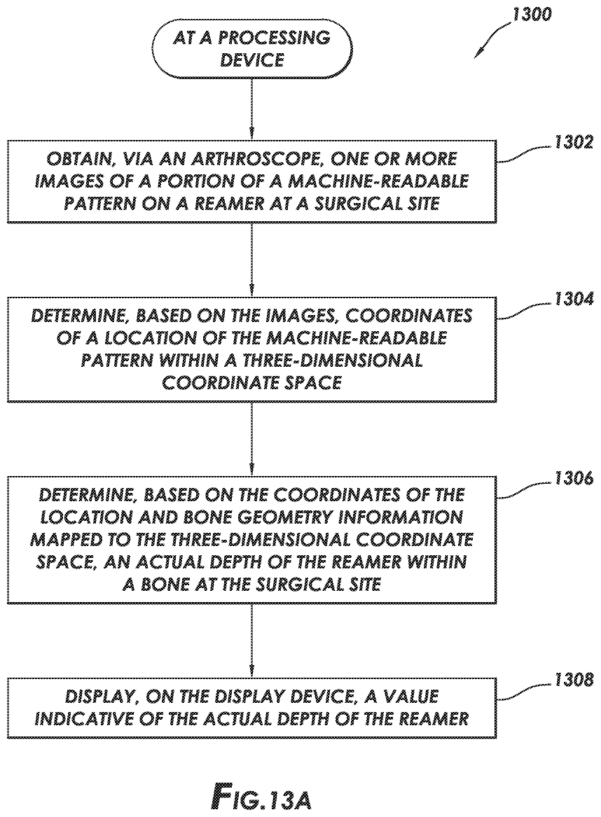

FIG. 12 shows a method of intraoperative determination of an actual depth of a reamer 800 within a bone 902, in accordance with at least some embodiments. In particular, the methods starts at block 1100. The method may be performed by one or more processors, such as included in the surgical controller 418, for example. The method may be implemented in computer instructions stored in one or more memories that are executed by the one or more processors.

At block 1202, the processor of the surgical controller 418 may display, on a display device 414, a value indicative of a parameter associated with a reamer 800 visible within a surgical site. The surgical controller 418 may read, using an arthroscope and a camera, a portion of a machine-readable pattern 802 on the reamer 800 visible within the surgical site. The surgical controller 418 may determine the value of the parameter of the reamer 800 within a bone based on the portion of the machine-readable pattern 802. In some examples, the parameter may include an actual depth of the reamer 800 within a bone 902, a diameter of the reamer 800, an identifier of the reamer 800, or some combination thereof.

At block 1204, the processor of the surgical controller 418 may determine whether the value indicative of the parameter satisfies a threshold value for the parameter. The threshold value may be configurable using the surgical controller 418. The threshold value may refer to a minimum value, a median value, a range of values, a maximum value, or the like.

At block 1206, responsive to determining the value indicative of the parameter satisfies the threshold value for the parameter, the surgical controller 418 may control operation of a drill associated with the reamer 800 at the surgical site by modifying an operating parameter of the drill. The operating parameter of the drill may pertain to a drilling rotational direction, a drilling rotational speed (e.g., revolutions per minute), an operating state (e.g., on or off), etc.

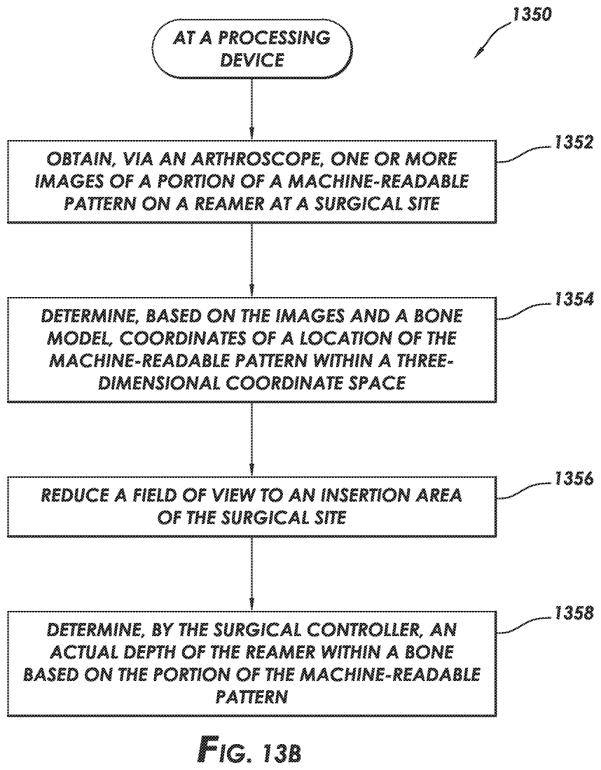

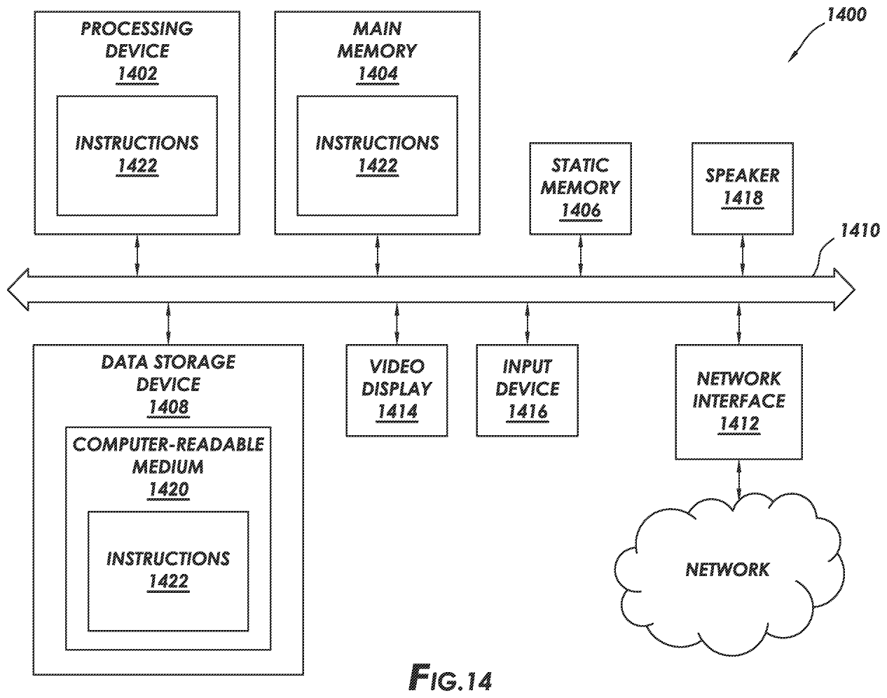

FIG. 13A shows a method 1300 of intraoperative determination of an actual depth of a reamer 800 within a bone 902, in accordance with at least some embodiments. In particular, the methods starts at block 1100. The method may be performed by one or more processors, such as included in the surgical controller 418, for example. The method may be implemented in computer instructions stored in one or more memories that are executed by the one or more processors.

At block 1302, the processor of the surgical controller 418 may obtain, via an arthrscope and a camera, one or more images of a portion of a machine-readable pattern 802 on a reamer 800 at a surgical site. At block 1304, the surgical controller 418 may determine, based on the images, coordinates of a location of the machine-readable pattern 802 within a three-dimensional coordinate space. At block 1306, the surgical controller 418 may determine, based on the coordinates of the location and bone geometry information mapped to the three-dimensional coordinate space, an actual depth of the reamer 800 within a bone 902 at the surgical site. At block 1308, the surgical controller 418 may display, on the display device 414, a value indicative of the actual depth of the reamer 800.

FIG. 13B shows a method 1350 of reducing a field of view to determine an actual depth of a reamer 800 within a bone 902, in accordance with at least some embodiments. In particular, the methods starts at block 1100. The method may be performed by one or more processors, such as included in the surgical controller 418, for example. The method may be implemented in computer instructions stored in one or more memories that are executed by the one or more processors.

At block 1352, the processor of the surgical controller 418 may obtain, via an arthroscope, one or more images of a portion of a machine-readable pattern on a reamer at a surgical site. At block 1354, the processor may determine, based on the one or more images and a bone model, coordinates of a location of machine-readable pattern within a three-dimensional coordinate space. At block 1356, the processor may reduce a field of view to an insertion area of the surgical site. The insertion area may refer to a location in the coordinate space where the processor determines the reamer is inserted into the bone and is drilling the hole in the bone. The processor may crop the considered area of images to a small radius around the identified insertion area. Accordingly, the memory size of the images may be reduced because the processor removed one or more images from consideration, thereby providing a technical improvement. At block 1358, the processor may determine, by the surgical controller, an actual depth of the reamer within the bone based on the portion of the machine-readable pattern.

FIG. 14 shows an example computer system 1400. In one example, computer system 1400 may correspond to the surgical controller 418, a tablet device within the surgical room, or any other system that implements any or all the various methods discussed in this specification. The computer system 1400 may be connected (e.g., networked) to other computer systems in a local-area network (LAN), an intranet, and/or an extranet (e.g., device cart 402 network), or at certain times the Internet (e.g., when not in use in a surgical procedure). The computer system 1400 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1400 includes a processing device 1402, a main memory 1404 (e.g., read-only memory (ROM), flash memory, dynamic random access memory (DRAM) such as synchronous DRAM (SDRAM)), a static memory 1406 (e.g., flash memory, static random access memory (SRAM)), and a data storage device 1408, which communicate with each other via a bus 1410.

Processing device 1402 represents one or more general-purpose processing devices such as a microprocessor, central processing unit, or the like. More particularly, the processing device 1402 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1402 may also be one or more special-purpose processing devices such as an application specific integrated circuit (ASIC), a field programmable gate array (FPGA), a digital signal processor (DSP), network processor, or the like. The processing device 1402 is configured to execute instructions for performing any of the operations and steps discussed herein. Once programmed with specific instructions, the processing device 1402, and thus the entire computer system 1400, becomes a special-purpose device, such as the surgical controller 418.

The computer system 1400 may further include a network interface device 1412 for communicating with any suitable network (e.g., the device cart 402 network). The computer system 1400 also may include a video display 1414 (e.g., display device 414), one or more input devices 1416 (e.g., a microphone, a keyboard, and/or a mouse), and one or more speakers 1418. In one illustrative example, the video display 1414 and the input device(s) 1416 may be combined into a single component or device (e.g., an LCD touch screen).

The data storage device 1408 may include a computer-readable storage medium 1420 on which the instructions 1422 (e.g., implementing any methods and any functions performed by any device and/or component depicted described herein) embodying any one or more of the methodologies or functions described herein is stored. The instructions 1422 may also reside, completely or at least partially, within the main memory 1404 and/or within the processing device 1402 during execution thereof by the computer system 1400. As such, the main memory 1404 and the processing device 1402 also constitute computer-readable media. In certain cases, the instructions 1422 may further be transmitted or received over a network via the network interface device 1412.

While the computer-readable storage medium 1420 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

### CLAUSES

1. A method comprising:
reading, by a surgical controller through an arthroscope, a portion of a machine-readable pattern on a reamer visible within a surgical site;
determining, by the surgical controller, an actual depth of the reamer within a bone based on the portion of the machine-readable pattern; and
displaying, by the surgical controller on a display device, a value indicative of the actual depth of the reamer within the bone.

2. The method of any clause herein, wherein determining the actual depth of the reamer further comprises:
extracting, by the surgical controller, a plurality of depth values from the portion of the machine-readable pattern; and
selecting only one of the plurality of depth values to be the actual depth.

3. The method of any clause herein, wherein reading the portion of the machine-readable pattern further comprises reading the portion of the machine-readable pattern comprising at least one annular stripe that circumscribes the reamer.

4. The method of any clause herein, wherein reading the portion of the machine-readable pattern further comprises reading the portion of the machine-readable pattern comprising at least one marking that only partially circumscribes the reamer.

5. The method of any clause herein, wherein reading the portion of the machine-readable pattern further comprises:
reading the portion of the machine-readable pattern at a first set of coordinates within a coordinate space;
receiving bone geometry information mapped within the coordinate space; and
determining, based on comparing the first set of coordinates and the bone geometry information, the at least one of the actual depth of the reamer and the trajectory of the reamer.

6. The method of any clause herein, wherein reading the portion of the machine-readable pattern further comprises reading the portion of the machine-readable pattern comprising one or more tick marks that are counted to determine the actual depth of the reamer within the bone.

7. The method of any clause herein, wherein reading the portion of the machine-readable pattern further comprises reading the portion of the machine-readable pattern comprising at least one marking including a barcode encoding the value of the actual depth of the reamer within the bone.

8. The method of any clause herein, wherein reading the portion of the machine-readable pattern further comprises:
identifying a gradient produced by the machine-readable pattern as the reamer rotates; and
determining, based on the gradient and a data source, the actual depth of the reamer within the bone.

9. The method of any clause herein, wherein the machine-readable pattern comprises a plurality of radial markings along a central axis of the reamer.

10. The method of any clause herein, wherein the plurality of radial markings are spaced apart by a distance representative of a value of the actual depth.

11. The method of any clause herein, wherein determining the actual depth of the reamer within the bone further comprises using a temporal sequence guide associated with a plurality of markings comprising the machine-readable pattern.

12. The method of any clause herein, further comprising:
prior to reading the portion of the machine-readable pattern on the reamer, reducing a revolutions per minute of the reamer from a first value to a second value.

13. The method of any clause herein, further comprising:
controlling, based on the actual depth of the reamer within the bone, operation of the reamer.

14. The method of any clause herein, further comprising:
receiving input, wherein the input pertains to the controlling the operation of the reamer.

15. The method of any clause herein, further comprising:
determining, by the surgical controller, a diameter of the reamer based on the portion of the machine-readable pattern; and
displaying, by the surgical controller on a display device, a value indicative of the diameter of the reamer.

16. The method of any clause herein, wherein a first portion of a shaft of the reamer including the machine-readable pattern has a smaller diameter than a second portion of the shaft of the reamer including cutting flutes.

17. The method of any clause herein, wherein the value indicative of the actual depth of the reamer within the bone is displayed in real-time or near real-time.

18. The method of any clause herein, further comprising:
displaying, by the surgical controller on the display device, a notification based on whether the value indicative of the actual depth of the reamer within the bone is within a threshold amount from a planned depth or has exceeded a planned depth.

19. The method of any clause herein, further comprising:
determining, based on the value of the actual depth, whether a planned depth of the reamer within the bone has been satisfied; and
responsive to determining the planned depth has been satisfied, controlling a drill operating the reamer by at least one of electronically stopping a drill operating the reamer and slowing down a speed of the drill.

20. The method of any clause herein, wherein a robotic arm supports positioning of the reamer.

21. A surgical controller comprising:
a processor configured to couple to a display device;
a memory coupled to the processor, the memory storing instructions that, when executed by the processor, cause the processor to:
receive one or more images of a visible portion of a reamer used at a surgical site for a bone, wherein the one or more images comprise a portion of a machine-readable pattern included on the reamer;
determine, based on the portion of the machine-readable pattern included on the reamer, a value indicative of a parameter associated with the reamer;
display, on a display device, the value indicative of the parameter.

22. The surgical controller of any clause herein, wherein the parameter comprises an actual depth of the reamer within the bone.

23. The surgical controller of any clause herein, wherein the parameter comprises a diameter of the reamer.

24. The surgical controller of any clause herein, wherein the instructions further cause the processor to:
determine, based on a value of the parameter, whether a planned value of the parameter associated with the reamer has been satisfied; and
responsive to determining the planned value of the parameter has been satisfied, electronically controlling operation of the drill associated with the reamer.

25. The surgical controller of any clause herein, wherein the one or more images are received from a camera associated with an arthroscope.

26. The surgical controller of any clause herein, wherein when the processor determines the value of the parameter, the instructions further cause the processor to:
identify a gradient produced by the machine-readable pattern as the reamer rotates; and
determine, based on the gradient and a data source, the value of the parameter of the reamer within the bone.

27. The surgical controller of any clause herein, wherein when the processor determines the value of the parameter, the instructions further cause the processor to:
read the portion of the machine-readable pattern at a first set of coordinates within a coordinate space;
receive bone geometry information of the bone mapped within the coordinate space; and
determine, based on comparing the first set of coordinates and the bone geometry information, the value of the parameter of the reamer.

28. The surgical controller of any clause herein, wherein when the processor determines the value of the parameter, the instructions further cause the processor to read the portion of the machine-readable pattern comprising one or more tick marks that are counted to determine the value of the parameter of the reamer.

29. The surgical controller of any clause herein, wherein when the processor determines the value of the parameter, the instructions further cause the processor to read the portion of the machine-readable pattern comprising at least one marking including a barcode encoding the value of the parameter of the reamer.

30. The surgical controller of any clause herein, wherein the processor:
receives, based on the value of the parameter, an input; and
controls, based on the input, operation of a drill associated with the reamer.

31. An intraoperative method comprising:
displaying, by a surgical controller on a display device, a value indicative of a parameter associated with a reamer visible within a surgical site;
determining whether the value indicative of the parameter satisfies a threshold value for the parameter; and
responsive to determining the value indicative of the parameter satisfies the threshold value for the parameter, controlling operation of a drill associated with the reamer at the surgical site by modifying an operating parameter of the drill.

32. The intraoperative method of any clause herein, further comprising:
reading, by the surgical controller through an arthroscope, a portion of a machine-readable pattern on the reamer visible within the surgical site;
determining, by the surgical controller, the value of the parameter of the reamer within a bone based on the portion of the machine-readable pattern.

33. The intraoperative method of any clause herein, wherein the parameter is an actual depth of the reamer within a bone.

34. The intraoperative method of any clause herein, wherein determining the value of the parameter of the reamer further comprises:
extracting, by the surgical controller, a plurality of depth values from the portion of the machine-readable pattern; and
selecting only one of the plurality of depth values to be the value of the actual depth.

35. The intraoperative method of any clause herein, wherein reading the portion of the machine-readable pattern further comprises reading the portion of the machine-readable pattern comprising at least one annular stipe that circumscribes the reamer.

36. The intraoperative method of any clause herein, wherein reading the portion of the machine-readable pattern further comprises reading the portion of the machine-readable pattern comprising at least one marking that only partially circumscribes the reamer.

37. The intraoperative method of any clause herein, wherein reading the portion of the machine-readable pattern further comprises:
reading the portion of the machine-readable pattern at a first set of coordinates within a coordinate space;
receiving bone geometry information mapped within the coordinate space; and
determining, based on comparing the first set of coordinates and the bone geometry information, at least one of the actual depth of the reamer and the trajectory of the reamer.

38. The intraoperative method of any clause herein, wherein reading the portion of the machine-readable pattern further comprises reading the portion of the machine-readable pattern comprising one or more tick marks that are counted to determine the value of the parameter of the reamer.

39. The intraoperative method of any clause herein, wherein reading the portion of the machine-readable pattern further comprises reading the portion of the machine-readable pattern comprising at least one marking including a barcode encoding the value of the parameter.

40. The intraoperative method of any clause herein, wherein reading the portion of the machine-readable pattern further comprises:
identifying a gradient produced by the machine-readable pattern as the reamer rotates; and
determining, based on the gradient and a data source, the value of the parameter of the reamer.

41. The intraoperative method of any clause herein, wherein the machine-readable pattern comprises a plurality of radial markings along a central axis of the reamer.

42. The intraoperative method of any clause herein, wherein the plurality of radial markings are spaced apart by a distance representative of a value of the parameter.

43. The intraoperative method of any clause herein, wherein determining the value of the parameter of the reamer further comprises using a temporal sequence guide associated with a plurality of markings comprising the machine-readable pattern.

44. The intraoperative method of any clause herein, further comprising determining the value of the parameter using infrared navigation of a drill an arthroscopic camera to triangulate a location of the reamer in three-dimensional space.

45. The intraoperative method of any clause herein, further comprising determining the value of the parameter using one or more three-dimensional positioning tools comprising a fiber optic shaped sensing (FOSS) system, an electromagnetic (EM) system, or some combination thereof.

46. A surgical controller comprising:
a processor configured to couple to a display device;
a memory coupled to the processor, the memory storing instructions that, when executed by the processor, cause the processor to:
obtain, via an arthroscope, one or more images of a portion of a machine-readable pattern on a reamer at a surgical site;
determine, based on the images, coordinates of a location of the machine-readable pattern within a three-dimensional coordinate space;
determine, based on the coordinates of the location and bone geometry information mapped to the three-dimensional coordinate space, an actual depth of the reamer within a bone at the surgical site; and
display, on the display device, a value indicative of the actual depth of the reamer.

47. The surgical controller of any clause herein, wherein the instructions further cause the processor to:
determine, based on the value of the actual depth of the reamer, whether a planned value of the actual depth associated with the reamer has been satisfied; and
responsive to determining the planned value of the actual depth has been satisfied, electronically controlling operation of a drill associated with the reamer.

48. The surgical controller of any clause herein, wherein the machine-readable pattern comprises at least one annular stripe that circumscribes the reamer.

49. The surgical controller of any clause herein, wherein the machine-readable pattern comprises at least one marking that only partially circumscribes the reamer.

50. The surgical controller of any clause herein, wherein the instructions further cause the processor to:
receive, based on the value of the actual depth of the reamer, an input to modify an operating parameter of a drill associated with the reamer; and
control operation of the drill by modifying the operating parameter of the drill.

1. A method comprising:
reading, by a surgical controller through an arthroscope, a portion of a machine-readable pattern on a reamer visible within a surgical site as the reamer rotates while drilling a bone and as the reamer translates into the bone;
determining, by the surgical controller, an actual depth of the reamer within the bone based on the portion of the machine-readable pattern visible during the drilling; and
displaying, by the surgical controller on a display device, a value indicative of the actual depth of the reamer within the bone.

2. The method of claim 1, wherein determining the actual depth of the reamer further comprises:
extracting, by the surgical controller, a plurality of depth values from the portion of the machine-readable pattern; and
selecting only one of the plurality of depth values to be the actual depth.

3. The method of claim 1, wherein reading the portion of the machine-readable pattern further comprises reading the portion of the machine-readable pattern selected from the group comprising at least one annular stripe that circumscribes the reamer and at least one marking that only partially circumscribes the reamer.

4. The method of claim 1, wherein reading the portion of the machine-readable pattern further comprises:
reading the portion of the machine-readable pattern at a first set of coordinates within a coordinate space;
receiving bone geometry information mapped within the coordinate space; and
determining, based on comparing the first set of coordinates and the bone geometry information, at least one of the actual depth of the reamer and the trajectory of the reamer.

5. The method of claim 1, wherein reading the portion of the machine-readable pattern further comprises reading the portion of the machine-readable pattern selected from the group comprising one or more tick marks that are counted to determine the actual depth of the reamer within the bone and at least one marking including a barcode encoding the value of the actual depth of the reamer within the bone.

6. The method of claim 1, wherein reading the portion of the machine-readable pattern further comprises:
identifying a gradient produced by the machine-readable pattern as the reamer rotates; and
determining, based on the gradient and a data source, the actual depth of the reamer within the bone.

7. The method of claim 1, wherein the machine-readable pattern comprises a plurality of radial markings along a central axis of the reamer.

8. The method of claim 7, wherein the plurality of radial markings are spaced apart by a distance representative of a value of the actual depth.

9. The method of claim 1, wherein determining the actual depth of the reamer within the bone further comprises using a temporal sequence guide associated with a plurality of markings comprising the machine-readable pattern.

10. The method of claim 1, further comprising:
prior to reading the portion of the machine-readable pattern on the reamer, reducing a revolutions per minute of the reamer from a first value to a second value.

11. The method of claim 1, further comprising:
controlling, based on the actual depth of the reamer within the bone, operation of the reamer.

12. The method of claim 11, wherein the machine-readable pattern is also human-readable.

13. The method of claim 1, further comprising:
determining, by the surgical controller, a diameter of the reamer based on the portion of the machine-readable pattern.

14. The method of claim 1, wherein a first portion of a shaft of the reamer including the machine-readable pattern has a smaller diameter than a second portion of the shaft of the reamer including cutting flutes.

15. The method of claim 1, further comprising:
displaying, by the surgical controller on the display device, a notification based on whether the value indicative of the actual depth of the reamer within the bone is within a threshold amount from a planned depth or has exceeded a planned depth.

16. The method of claim 1, further comprising:
determining, based on the value of the actual depth, whether a planned depth of the reamer within the bone has been satisfied; and
responsive to determining the planned depth has been satisfied, controlling a drill operating the reamer by at least one of electronically stopping a drill operating the reamer and slowing down a speed of the drill.

17. A surgical controller comprising:
a processor configured to couple to a display device;
a memory coupled to the processor, the memory storing instructions that, when executed by the processor, cause the processor to:
receive one or more images of a visible portion of a reamer used at a surgical site for a bone, wherein the one or more images comprise a portion of a machine-readable pattern included on the reamer as the reamer rotates while drilling the bone and as the reamer translates into the bone;
determine, based on the portion of the machine-readable pattern included on the reamer, a value indicative of a parameter associated with the reamer visible during the drilling; and
display, on the display device, the value indicative of the parameter.

18. The surgical controller of claim 17, wherein the parameter is selected from the group comprising an actual depth of the reamer within the bone and a diameter of the reamer.

19. The surgical controller of claim 17, wherein the instructions further cause the processor to:
determine, based on a value of the parameter, whether a planned value of the parameter associated with the reamer has been satisfied; and
responsive to determining the planned value of the parameter has been satisfied, electronically controlling operation of a drill associated with the reamer.

20. The surgical controller of claim 17, wherein the one or more images are received from a camera associated with an arthroscope.

21. The surgical controller of claim 17, wherein when the processor determines the value of the parameter, the instructions further cause the processor to:
identify a gradient produced by the machine-readable pattern as the reamer rotates; and
determine, based on the gradient and a data source, the value of the parameter of the reamer within the bone.

22. The surgical controller of claim 17, wherein when the processor determines the value of the parameter, the instructions further cause the processor to:
read the portion of the machine-readable pattern at a first set of coordinates within a coordinate space;
receive bone geometry information of the bone mapped within the coordinate space; and
determine, based on comparing the first set of coordinates and the bone geometry information, the value of the parameter of the reamer.

23. The surgical controller of claim 17, wherein when the processor determines the value of the parameter, the instructions further cause the processor to read the portion of the machine-readable pattern selected from the group comprising one or more tick marks that are counted to determine the value of the parameter of the reamer and at least one marking including a barcode encoding the value of the parameter of the reamer.

24. The surgical controller of claim 17, wherein the processor:
receives, based on the value of the parameter, an input; and
controls, based on the input, operation of a drill associated with the reamer.

25.-40. (canceled)
